(19) United States
(12) Patent Application Publication
(10) Pub. No.: US 2008/0038151 A1
(43) Pub. Date: Feb. 14, 2008

(54) PRESERVING APPARATUS HAVING MULTIPLE SURFACE ELECTRODE STRUCTURE

(75) Inventors: Min Sun KIM, Hwaseong-si (KR); Seong Gu KIM, Pyeongtaek-si (KR)

Correspondence Address: CANTOR COLBURN, LLP, 55 GRIFFIN ROAD SOUTH, BLOOMFIELD, CT, 06002, US

(73) Assignee: SAMSUNG ELECTRONICS CO., LTD., Suwon-si (KR)

(21) Appl. No.: 11/772,531

(22) Filed: Jul. 2, 2007

(30) Foreign Application Priority Data
Aug. 11, 2006 (KR) 10-2006-0076061

(51) Int. Cl. A61L 2/03; C25B 11/04

(52) U.S. Cl. 422/22; 204/280

(57) ABSTRACT

A preserving apparatus has a multiple surface electrode structure that can keep an object disposed in a preserving apparatus fresh by disrupting an electrochemical balance in a microorganism and controlling a flow of ions in the microorganism from multiple surfaces. The preserving apparatus has a multiple surface electrode structure that includes a multiple surface electrode unit having at least one anode and cathode facing each other and located in a housing member of a preserving unit that stores an object disposed in the preserving apparatus; and a power supply unit that forms an electric field between the anode and the cathode by supplying a voltage to the anode and the cathode; wherein the power supply unit sequentially or randomly supplies the voltage to the at least one anode and cathode.

### CROSS-REFERENCE TO RELATED APPLICATION

This application claims the benefit of Korean Patent Application No. 10-2006-0076061, filed on Aug. 11, 2006, in the Korean Intellectual Property Office, the disclosure of which is hereby incorporated by reference in its entirety.

### BACKGROUND OF THE INVENTION

1. Field of the Invention

The present invention relates to a preserving apparatus having a multiple surface electrode structure. More particularly, the present invention relates to a preserving apparatus that can prevent a microorganism from proliferating while contained in an object that is disposed in the preserving apparatus. The preserving apparatus controls the growth and proliferation of microorganisms by controlling or limiting the flow of charged particles in the microorganism by exposing the microorganism to an electric field, which is applied in more than one direction via multiple electrodes.

2. Description of Related Art

Microorganisms plays an important role in the lives of living beings. In particular, the microorganism can be the cause of an infectious disease. Microorganisms can also be advantageous to a human body by preventing the spread of the disease. Microorganisms can pollute the body of a living being from the air, water, via animals, food and the like. It is important to effectively control the growth of such polluting microorganisms to facilitate an improvement in the quality of human life. Controlling such microorganisms is useful in various fields of industry and various methods have been developed in various fields to do so. However, currently available methods are not applicable to all of the various types of microorganisms, therefore a new method to control all types of microorganisms, especially those present in food, is desired.

Depending upon the types of food, various methods are used to control the microorganisms present in the food and thereby extend the expiry dates of the food. However, there is a limit in terms of the usage of these currently available methods to the home refrigerator and new methods are needed to meet the needs in the food storage at home.

The cell membrane of all organisms comprises free ions, e.g., K+, Na+, Cr, Ca²⁺. The free ions functions as follows: i) control a volume of the cell by generating an osmotic pressure that controls the entrance and the amount of water into the cell; ii) plays a key role in other metabolic processes, such as a transduction process; iii) generates a strong electric field of 10⁷ V/m between the cell membranes. An ion flux via the cell membrane is generated by a concentration of free ions, which exists within the cell membrane and the application of a voltage to the cell.

The difference in the electrical potential across the cell membrane of an organism is due to the sum of contributions of all free ions present in the cell. When an external electrical field is supplied to the organism, two possible results may occur. First, when the external electric field is static, a polarization in the cell has a predetermined direction and a size, and when the external electric field oscillates, the free ions are forced to vibrate. Second, when the external electric field is harmonic or alternating, the external electric field functions as a periodical force not only on all ions present in a plasma membrane of the organism but also on all ions present in a protein channel present in the organism. The alternating external electrical field promotes all free ions to vibrate. When an amplitude of the oscillation of the ions is greater than a predetermined threshold, the oscillating ions may give an erroneous signal of “open and close signal” of the protein channel, i.e., a voltage channel. This phenomenon may disrupt an electrochemical balance of the cell membrane, which subsequently may hinder the entire function of the cell.

The method of maintaining of food freshness using an electric field is not popular because of a number of problems, one of which is the lack of reproducibility. Maintaining food freshness by using an alternating electric field that destroys undesirable living organisms is useful in refrigeration devices that need to preserve food for long periods of time. However, the lack of reproducibility is an undesirable hindrance to achieving such long term food preservation. This lack of reproducibility is possibly due to the adjustment ability of the microorganism to an external electric field and to the fact that the target ion channels are not arranged perpendicular to the electric field due to the three-dimensional shape of microorganism (membrane). To overcome these, electric fields with more than one direction should be applied for the growth of microorganisms to be affected as a result. Therefore, multiple electrodes will be employed to produce electric fields with more than one direction.

FIG. 1 illustrates a prior art preserving apparatus that uses an electrode structure to form an electric field.

Referring to FIG. 1, the prior art preserving apparatus 100 has an electrode structure that can form an electric field. The apparatus includes a shelf 110 having a metal electrode and a power supply unit 120 to supply a voltage to the electrode to form an electric field.

In the prior art preserving apparatus 100, an object whose freshness is desired to be preserved is disposed in the preserving apparatus on the shelf 110. As shown in the FIG. 1, the shelf 110 is connected to an anode of the power supply unit 120 by a connector 130. A positive charge is distributed on a surface of the metal electrode of the shelf 110, and accordingly, an electric field is formed. Since the main electric field is vertically formed in one direction as determined by the structure, the aforementioned external electric field may not prevent a microorganism from proliferating in an object that is contained in the preserving apparatus.

### BRIEF SUMMARY

The invention is described more fully hereinafter with reference to the accompanying drawings, in which embodiments of the invention are shown. This invention, however, should not be construed as limited to the embodiments set forth herein. Rather, these embodiments are provided to convey the scope of the invention to those skilled in the art. In the drawings, the size and relative sizes of elements and regions may be exaggerated for clarity.

It will be understood that when an element or layer is referred to as being “on,” “interposed,” “disposed,” or “between” another element or layer, it can be directly on, interposed, disposed, or between the other element or layer or intervening elements or layers may be present.

It will be understood that, although the terms first, second, third, and the like may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, first element, component, region, layer or section discussed below could be termed second element, component, region, layer or section without departing from the teachings of the present invention.

As used herein, the singular forms “a,” “an” and “the” are intended to comprise the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein. In one embodiment, a preserving apparatus has a multiple surface electrode structure that can keep an object in the preserving apparatus fresh.

In another embodiment, the preserving apparatus has a multiple surface electrode structure that can disrupt an electrochemical balance of a microorganism by controlling the flow of ions from multiple surfaces in the microorganism.

In yet another embodiment, the preserving apparatus has a multiple surface electrode structure that follows a mechanism having an effect on a biochemical environment in a cell of a microorganism.

In yet another embodiment, there is provided a to a preserving apparatus having a multiple surface electrode structure including: a multiple surface electrode comprising an anode and a cathode facing each other and located in a housing member of the preserving apparatus; and a power supply unit forming an electric field between the anode and the cathode by supplying a voltage to the anode and the cathode; wherein the power supply unit sequentially or randomly supplies the voltage to the anode and cathode.

As detailed above, the application of an external alternating electric field to a microorganism promotes a disruption of the ionic fields in the cell thereby destroying the microorganism. Specifically, when the amplitude of the external electric field is greater than a predetermined amplitude it has a detrimental effect on the function of the cell, namely it becomes difficult for the organism to maintain a desired membrane potential, which in turn disrupts an electrochemical balance in the cell.

An oscillating ion such as K+ and Ca²⁺, under a forced vibration places a mechanical pressure or a force on a plasma membrane, and has the effect on the opening and closing of ion channels, present within the organism. Consequently, as a result of the forced vibration, abnormal gating of the ion channels occurs. This abnormal gating also has a detrimental effect on the cell and disrupts the electrochemical balance in the cell.

Additional and/or other aspects and advantages will be set forth in part in the description which follows and, in part, will be obvious from the description, or may be learned by practice of the invention.

### BRIEF DESCRIPTION OF THE DRAWINGS

The above and/or other aspects and advantages will become apparent and more readily appreciated from the following detailed description, taken in conjunction with the accompanying drawings of which:

FIG. 1 illustrates a prior art preserving apparatus having an electrode structure that facilitates the formation of an electric field;

FIG. 2 is an exemplary cross-sectional diagram illustrating a structure of a preserving apparatus having a multiple surface structure according to an exemplary embodiment;

FIGS. 3A through 3F each illustrate a simulation result of the distribution and the direction of an electric field according to the supply of the electric field at each electrode of the preserving apparatus of FIG. 2;

FIG. 4 is an exemplary cross-sectional diagram illustrating the electrode of the multiple electrodes according to an exemplary embodiment;

FIG. 5 illustrates the power supply unit that supplies a voltage for forming an electric field in the preserving apparatus having a cube electrode according to an exemplary embodiment;

FIGS. 6A through 6E illustrates multiple electrodes according to an exemplary embodiment; and

FIGS. 7A and 7B illustrates a distance controller to control a distance between electrodes of the multiple electrodes according to another exemplary embodiment.

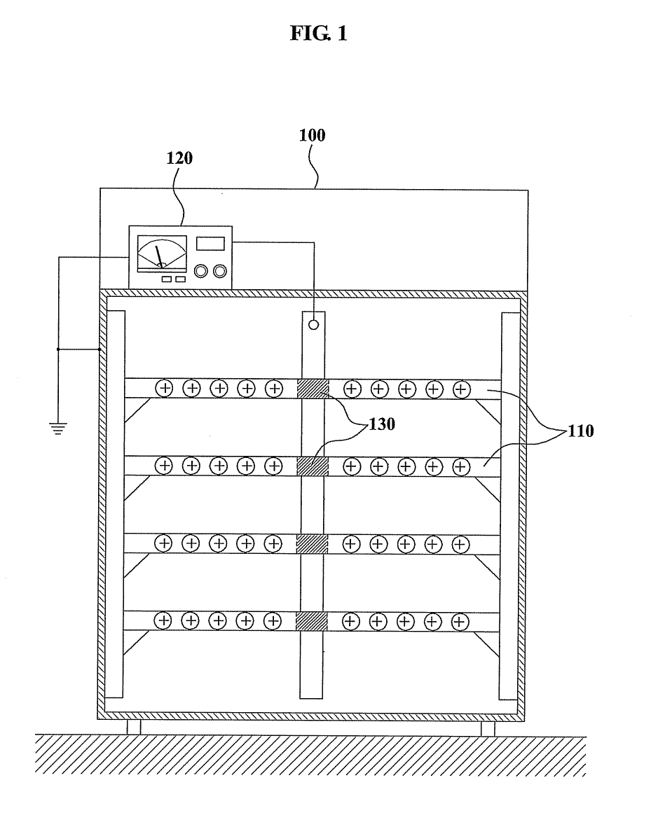

### DETAILED DESCRIPTION OF EMBODIMENTS

Reference will now be made in detail to exemplary embodiments, examples of which are illustrated in the accompanying drawings, wherein like reference numerals refer to the like elements throughout. The exemplary embodiments are described below in order to explain various details by referring to the figures.

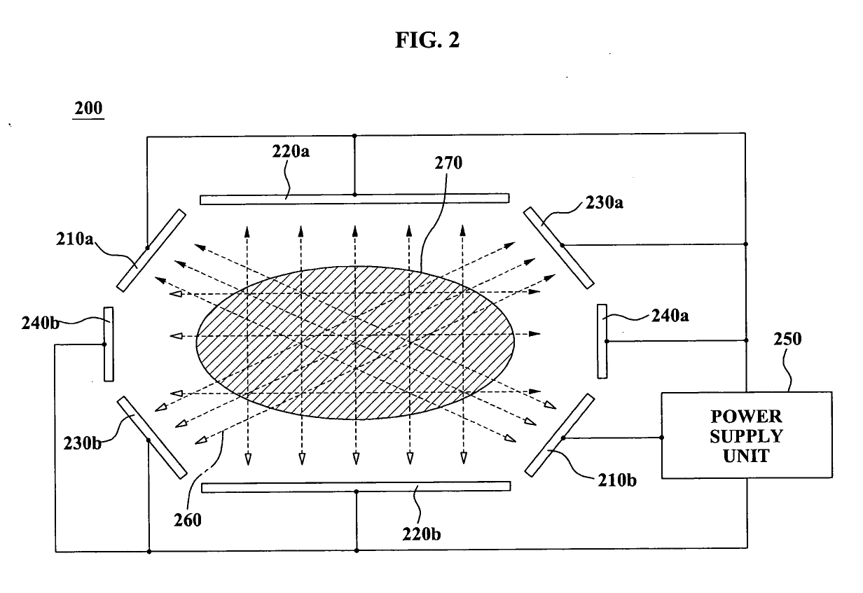

FIG. 2 is a cross-sectional diagram illustrating a structure of a preserving apparatus having a multiple surface structure according to various exemplary embodiments. Referring to FIG. 2, the preserving apparatus 200 having the multiple surface structures according to the exemplary embodiment of the present invention includes multiple electrode units and a power supply unit 250.

The multiple electrode units 210, 220, 230, and 240 have least one anode and cathode are facing each other, and are located in a housing member of a preserving unit storing an object 270 the preserving apparatus 200. In the multiple electrode units 210, 220, 230, and 240, anodes 210a, 220a, 230a, and 240a, and cathodes 210b, 220b, 230b, and 240b face each other, and when the power supply unit 250 supplies an alternating power, the anodes and the cathodes may switch their roles. In other words, an anode 210a may function as a cathode 210a, upon reversal of the applied electrical field. When the anodes and the cathodes switch their roles, the direction of the electric field 260 between the two becomes inversed. A case of when the direction of the electric field 260 inverses is illustrated in FIG. 2, where the direction of the electric field 260 is distinguished by the color of arrows, i.e. black and white. The multiple electrode units 210, 220, 230, and 240 are made of any conductive material. The conductive materials may comprise metals, electrically conductive metal oxides or electrically conducting polymers. Examples of suitable metals are gold (Au), silver (Ag), nickel (Ni), chromium (Cr), copper (Cu), or the like, or a combination comprising at least one of the foregoing. Examples of suitable electrically conducting metal oxides are tin oxide, antimony tin oxide, indium tin oxide (ITO), or the like, or a combination comprising at least one of the foregoing metal oxides. Examples of suitable conducting polymers are polypyrrole, polythiophene, polyaniline, polyacetylene, or the like, or a combination comprising at least one of the foregoing conducting polymers.

The power supply unit 250 forms an electric field of a predetermined size between the anodes 210a, 220a, 230a, and 240a, and cathodes 210b, 220b, 230b and 240b by supplying a voltage to the anodes and the cathodes. Specifically, the power supply unit 250 may sequentially or randomly supply the voltage to at least one of the anodes 210a, 220a, 230a, and 240a and cathodes 210b, 220b, 230b and 240b. Also, the power supply unit 250 may supply a voltage which is any one of a direct current (DC) voltage, an alternating current (AC) voltage, or a voltage synthesized from a combination of the DC voltage and the AC voltage. Specifically, the biochemical balance in a microorganism may be disrupted by increasing the amplitude of the applied external electric field beyond that which the microorganism can withstand. In one embodiment, the electric field is superposed on at least one electric field, the at least one electric field being having an electric field strength of less than or equal to 100 kV/m, and has a frequency of less than or equal to 100 MHz. The electric field of less than 100 kV/m and the frequency of less than or equal to 100 MHz enables the preserving apparatus 200 to control the microorganism.

The application of the external alternating field can disrupt or control an electrochemical balance in the microorganism by controlling the flow of an ion thereby effecting the biochemical environment in the cells of the microorganism.

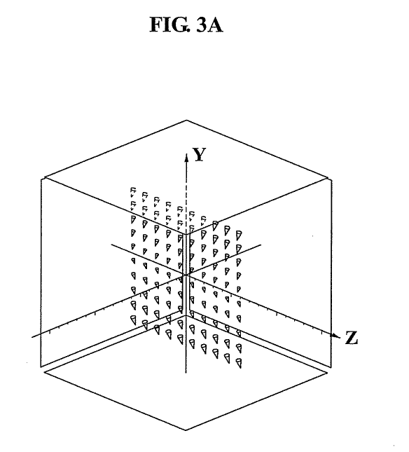

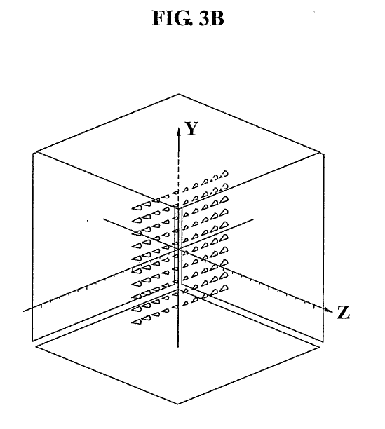

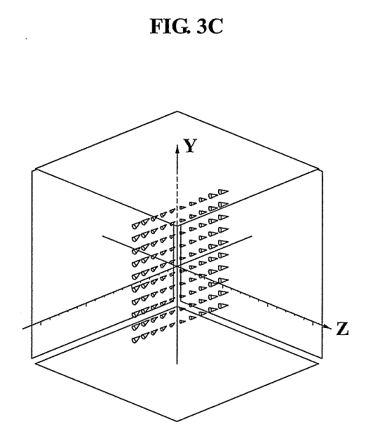

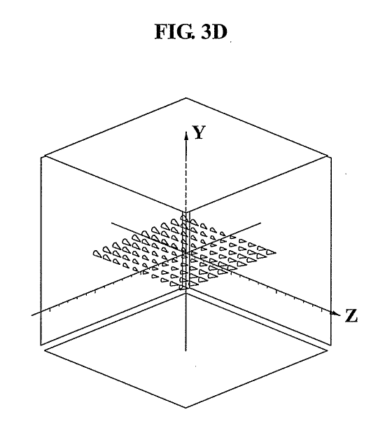

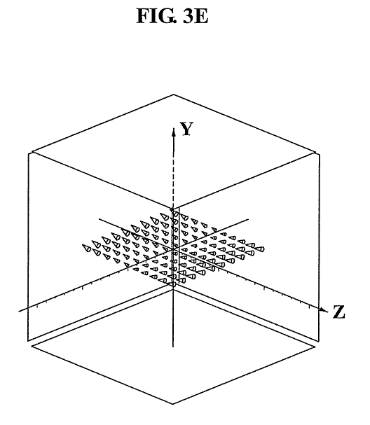

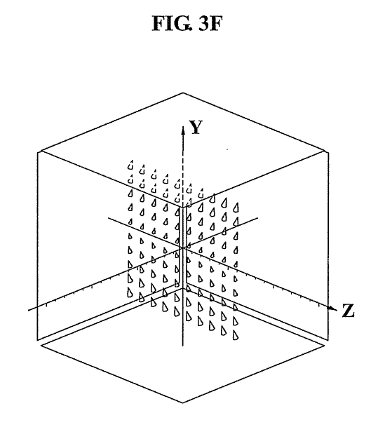

FIGS. 3A through 3F illustrate the results of a simulation wherein the distribution and direction of an electric field in the preserving apparatus of FIG. 2 were studied in a preservation apparatus having a multiple surface structure, i.e. a cube structure.

Referring to FIGS. 3A through 3F, the size of the cones in each of the FIGS. 3A through 3F designates the size of the electric field, and the direction from the bottom to the apex of the cone designates the direction of the electric field. As described above, the electric field is respectively supplied to a pair of each electrode in FIGS. 3A through 3F. As illustrated in the FIGS. 3A through 3F, since the voltage is sequentially or randomly supplied each pair of electrodes by the power supply unit 250 of FIG. 2, ion channels in the microorganism may be multilaterally controlled when an object is placed in a housing member of the preserving apparatus 200 of FIG. 2.

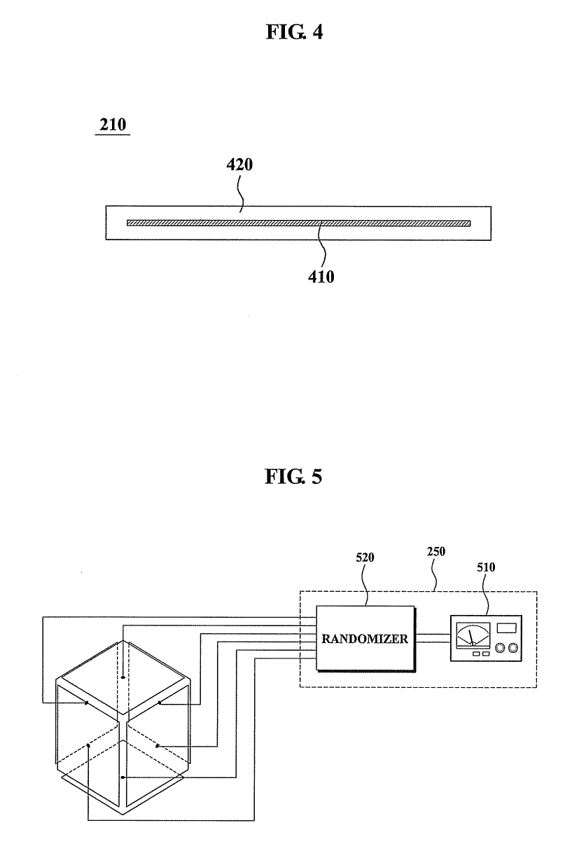

FIG. 4 is a cross-sectional diagram illustrating each electrode of the multiple electrode unit 210 of FIG. 2 according to an exemplary embodiment of the present invention.

Referring to FIG. 4, the multiple electrode unit 210 of FIG. 2 includes an electrode 410 and a dielectric material 420.

The electrode 410 is made of the electrically conductive materials described above. Also, an electrode, which is coated by a metal whose ionization tendency is less than a metal, may be formed on the metal surface of the electrode 410.

The dielectric material 420 may correspond to any electrically insulating material having an electrical resistivity greater than or equal to about 10¹⁵ ohm-cm.

Examples of suitable electrically insulating materials are glass, alumina, teflon, TiO₂, BaTiO₃, polyimide, polystyrene, polymethylmethacrylate (PMMA), polyvinylalcohol, polyvinylphenol, polycarbonate, polyester, polyolefin, benzocylobutene (BCB), parylene—C, 2-amino-4,5-imidazoledicarbonitrile, metal phthalocyanine, LiF, silicon dioxide, silicon nitride derivative, aluminum oxide (Al₂O₃), Ta₂O₅, AlN, AlON, La₂O₅, BaZrTiO₃ and PbZrTiO₃, or the like, or a combination comprising at least one of the foregoing electrically insulating materials. The multiple electrode units are not limited to the structure that is illustrated in FIG. 4.

FIG. 5 illustrates the power supply unit to supply a voltage for forming an electric field in the preserving apparatus having a cube shaped electrode (similar to those depicted in FIGS. 3A through 3F) according to an exemplary embodiment of the present invention.

Referring to FIG. 5, the power supply unit 250 includes a power supply 510 and a randomizer 520. The power supply 510 supplies a voltage, which is any one of a pulsed voltage, an AC voltage, a DC voltage, or a voltage synthesized from at least one of the DC voltage and the AC voltage. The randomizer 520 sequentially or randomly supplies a voltage to each electrode unit of the preserving apparatus having the cube shaped electrode. A voltage may be multilaterally supplied to the cube shaped electrode by the power supply unit 500.

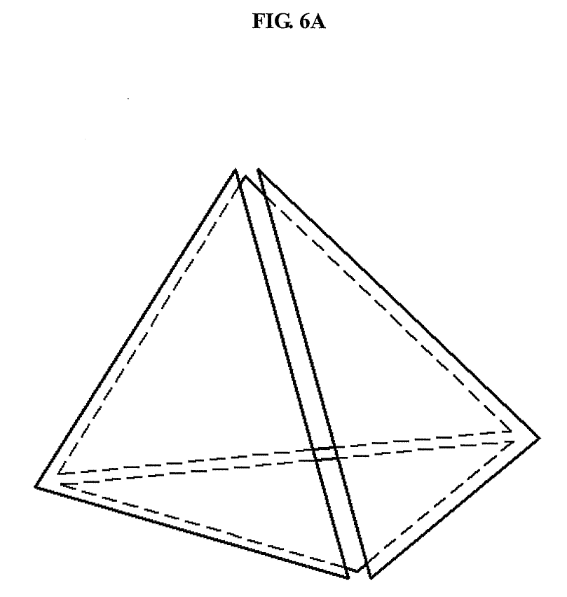

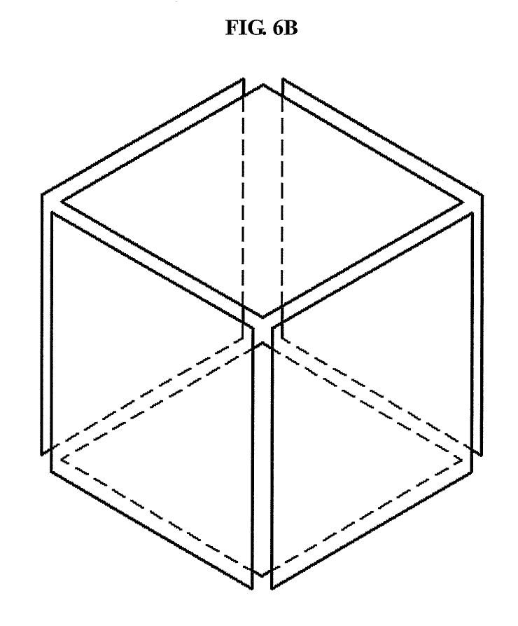

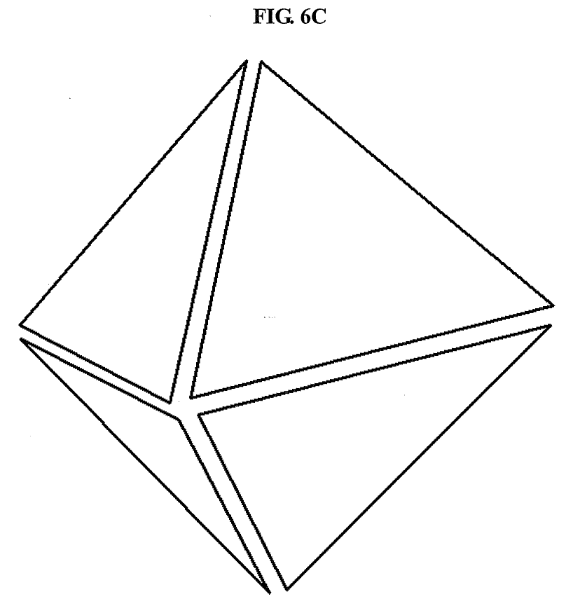

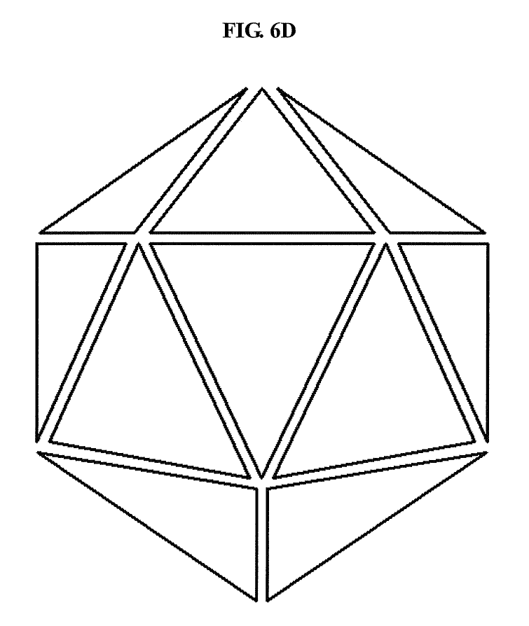

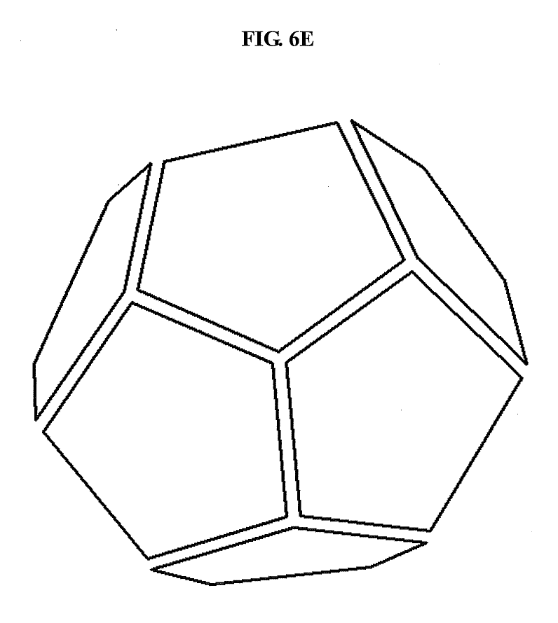

FIGS. 6A through 6E illustrate different designs for the multiple electrode units according to another exemplary embodiment.

Referring to FIGS. 6A through 6E, the multiple electrode units includes regular polyhedrons, e.g. a tetrahedron (FIG. 6A), a cube (FIG. 6B), an octahedron (FIG. 6C), a dodecahedron (FIG. 6D), an icosahedron (FIG. 6E). The preservation apparatus may also have an irregular shape (not shown). Also, the multiple electrode units may be a multiple electrode that can supply an electric field from various directions even when has an irregular shape.

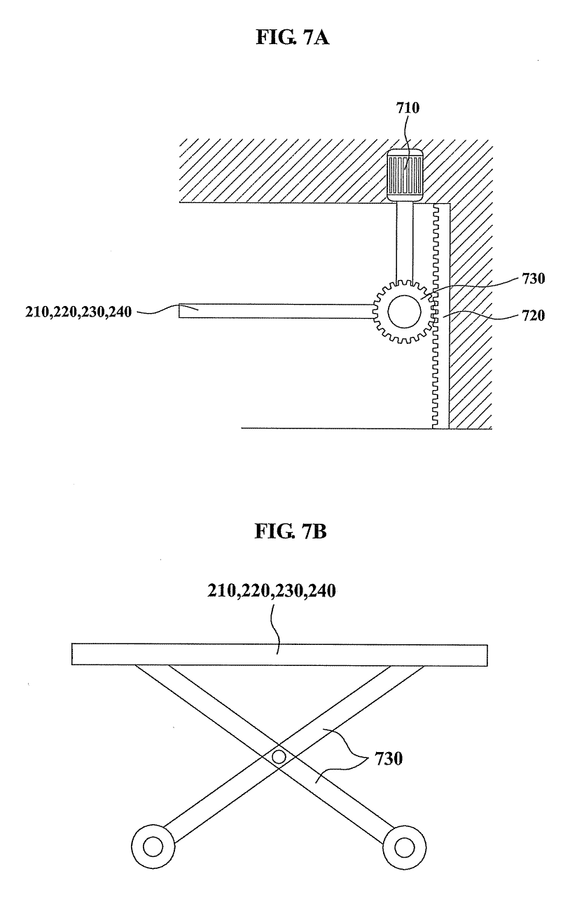

FIGS. 7A and 7B illustrate a distance controller to control a distance between electrodes of the preservation apparatus according to another exemplary embodiment.

FIGS. 7A and 7B illustrate one multiple electrode unit from the multiple electrode units 210, 220, 230, and 240 of FIG. 2 wherein the distance controller may be used to control the respective distances between the electrodes 210a and 210b, 220a and 220b, 230a and 230b, and 240a and 240b. Referring to FIG. 7A, the distance controller according to the exemplary embodiment includes a motor 710, a rack 720 and a pinion 730. The distance between electrodes of the multiple electrode units 210, 220, 230 and 240 may be controlled since the pinion 730 rotates to engage with the rack 720 by connecting with the motor 710. The distance controller is applied to all of the multiple electrode units 210, 220, 230, and 240 although only one multiple electrode is illustrated in FIG. 7A. Also, according to another embodiment, the multiple electrode units may be configured to automatically ascend or descend by mounting a sensor and measuring the height of an object to be placed on a housing member of the preserving apparatus, and the distance between the electrodes of the multiple electrode units may be controlled since a user controls the distance using a control button without the sensor.

Also, referring to FIG. 7B, the distance controller according to the another embodiment includes a shelf which is supported by an X shaped link 740, the X shaped link 740 having a groove and a protrusion. The location of the X shaped link 740 may be vertically controlled by a control button since the link is connected to a motor.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A preserving apparatus having a multiple surface electrode structure, the apparatus comprising:
a multiple surface electrode unit having an anode and a cathode facing each other and located in a housing member of a preserving unit storing an object of the preserving apparatus; and
a power supply unit forming an electric field between the anode and the cathode by supplying a voltage to the anode and the cathode; the power supply unit sequentially or randomly supplying the voltage to the anode and the cathode.

2. The apparatus of claim 1, wherein the power supply unit supplies a direct current (DC) voltage, an alternating current (AC) voltage, or a voltage synthesized comprising the DC voltage and the AC voltage.

3. The apparatus of claim 1, wherein the electric field is superposed on at least one electric field, the at least one electric field being less than or equal to 100 kV/m and having a frequency of less than or equal to 100 MHz.

4. The apparatus of claim 1, wherein the multiple surface electrode unit comprises a an electrically conducting material, wherein the electrically conducting material comprises a metal, an electrically conducting metal oxide, an electrically conducting polymer, or a combination comprising at least one of the foregoing electrically conducting materials.

5. The apparatus of claim 4, wherein the metal is gold, silver, nickel, chromium, copper, or a combination comprising at least one of the foregoing metals.

6. The apparatus of claim 4, wherein the electrically conducting metal oxide is indium tin oxide, tin oxide, antimony tin oxide, or a combination comprising at least one of the foregoing metal oxides.

7. The apparatus of claim 4, wherein the electrically conducting polymer is a polypyrrole, polyaniline, polythiophene, polyacetylene or a combination comprising at least one of the foregoing electrically conducting polymers.

8. The apparatus of claim 1, wherein the multiple surface electrode unit comprises plate-type electrode that comprises gold, silver, nickel, chromium, copper, indium tin oxide, antimony tin oxide, tin oxide, and is surrounded by a dielectric material.

9. The apparatus of claim 8, wherein the dielectric material is glass, alumina, teflon, TiO₂, BaTiO₃, polyimide, polystyrene, polymethylmethacrylate, polyvinylalcohol, polyvinylphenol, polycarbonate, polyester, polyolefin, benzocylobutene (BCB), parylene—C, 2-amino-4,5-imidazoledicarbonitrile, metal phthalocyanine, an organic selected from a derivative of one of the above organic compounds, LiF, silicon dioxide, silicon nitride and its derivatives, aluminum oxide (Al₂O₃), Ta₂O₅, AlN, AlON, La₂O₅, BaZrTiO₃, PbZrTiO₃, an inorganic selected from derivatives of one of the above inorganic compoundsor a combination comprising at least one of the foregoing dielectric materials.

10. The apparatus of claim 1, wherein the apparatus further comprises a distance controller to control a distance between the anode and cathode.

11. The apparatus of claim 1, wherein the apparatus comprises a plurality of anodes and cathodes that are disposed to be parallel to one another.

12. A method for maintaining freshness comprising:
disposing an object whose preservation is desired upon a shelf; the shelf comprising a plurality of anodes and cathodes that are in electrical communication with a power supply unit; and
subjecting the anode, the cathode and the object to an alternating electrical field.
